(19) United States
(12) Patent Application Publication
(10) Pub. No.: US 2013/0226199 A1
(43) Pub. Date: Aug. 29, 2013

(54) OMNIDIRECTIONAL CLOSURE CLIP

(71) Applicant: Boston Scientific Scimed, Inc., (US)

(72) Inventors: Colby HARRIS, Weston, MA (US); Gary Jordan, Litchfield, NH (US)

(73) Assignee: Boston Scientific Scimed, Inc., Maple Grove, MN (US)

(21) Appl. No.: 13/777,159

(22) Filed: Feb. 26, 2013

Related U.S. Application Data
(60) Provisional application No. 61/604,046, filed on Feb. 28, 2012.

(51) Int. Cl. A61B 17/10

(52) U.S. Cl. CPC A61B 17/10
USPC 606/142

(57) ABSTRACT

A tissue clipping device includes a capsule extending from a proximal end to a distal end and including a lumen extending therethrough, a clip including at least three arms biased toward an open configuration and connected to one another at proximal ends thereof, the arms separated from one another circumferentially about a centerline of the clip and the proximal ends of the arms slidable within the lumen of the capsule to move the clip between the open configuration, in which distal ends of the arms are separated from one another to receive tissue therebetween and a closed configuration in which distal ends of the arms are moved toward one another to grip tissue in combination with a tension member releasably coupling the clip to a proximal end of the device which remains accessible to a user, to control movement of the clip between the open and the closed configurations.

### PRIORITY CLAIM

This application claims the priority to the U.S. Provisional Application Ser. No. 61/604,046, entitled “Omnidirectional Closure Clip” filed on Feb. 28, 2012. The specification of the above-identified application is incorporated herewith by reference.

### BACKGROUND

Pathologies of the gastro-intestinal (GI) system, the biliary tree, the vascular system and other body lumens and hollow organs are often treated through endoscopic procedures, many of which require active and/or prophylactic hemostasis. Tools for deploying hemostatic clips via endoscopes are often used to stop internal bleeding by clamping together edges of wounds or incisions. These clips grasp tissue surrounding a wound holding edges of the wound together until natural healing processes have closed the wound. Many current clips include two arms moved toward one another to grip tissue therebetween. In some cases the clip arms must be positioned in a particular angular orientation to grasp the target tissue edges. Thus, application of the clips requires that they be rotatable by a user. However, it has proven difficult with certain clips to transmit the torque required to rotate clips over the length of the flexible member which connects the clip to the actuator. This difficulty is especially pronounced when the clip device extends along a tortuous path from the actuator to the target tissue. Alternatively, in some cases, a user may be required to utilize multiple clips to pull tissue together from multiple directions.

### SUMMARY OF THE INVENTION

The present invention relates to a tissue clipping device which includes a capsule extending from a proximal end to a distal end and including a lumen extending therethrough and a clip including a plurality of arms (e.g., four arms) biased toward an open configuration and connected to one another at proximal ends thereof. The arms are separated from one another circumferentially about a centerline of the clip and the proximal ends thereof may be slidable within the lumen of the capsule to move the clip between the open configuration, in which distal ends of the arms are separated from one another to receive tissue therebetween and a closed configuration, in which the distal ends of the arms are drawn toward one another to grip tissue therebetween. The device also comprises a tension member releasably coupling the clip to a proximal end of the device which, during use, remains accessible to a user, to control movement of the clip between the open and the closed configurations.

### BRIEF DESCRIPTION OF THE DRAWINGS

FIG. 1 shows a perspective view of a clipping device according to an exemplary embodiment of the present invention;

FIG. 2 shows a side view of a control wire of the clipping device of FIG. 1;

FIG. 3 shows a perspective view of a constraint tube of the clipping device of FIG. 1;

FIG. 4 shows a perspective view of a clip and capsule of the clipping device of FIG. 1, in an open configuration;

FIG. 5 shows a view from a distal end of the clip and capsule of FIG. 4;

FIG. 6 shows a perspective view of the clip and capsule of FIG. 1, in a closed and locked configuration;

FIG. 7 shows a perspective view of the clip of the clipping device of FIG. 1;

FIG. 8 shows an enlarged perspective view of a proximal end of the clip of FIG. 7;

FIG. 9 shows a longitudinal cross-sectional view of a capsule and bushing of the clipping device of FIG. 1;

FIG. 10 shows a cross-sectional view of a clipping device according to another exemplary embodiment of the present invention;

FIG. 11 shows a lateral cross-sectional view of the device of FIG. 10 along the lines A-A, in an undeployed configuration;

FIG. 12 shows a longitudinal cross-sectional view of the device of FIG. 10, in a deployed configuration;

FIG. 13 shows a perspective view of a clipping device according to yet another exemplary embodiment of the present invention;

FIG. 14 shows a longitudinal cross-sectional view of a clip locking into a capsule according to the device of FIG. 13; and

FIG. 15 shows a perspective view of the clip according to the device of FIG. 14.

### DETAILED DESCRIPTION

The present invention may be further understood with reference to the following description and the appended drawings, wherein like elements are referred to with the same reference numerals. The present invention relates to an endoscopic devices and, in particular, clipping devices. Exemplary embodiments of the present invention describe a clip including multiple clip arms to facilitate an omnidirectional closure about a target tissue such that a user is not required to rotate the clip to achieve a particular orientation of the clip arms relative to a target tissue. It should be noted that the terms “proximal” and “distal,” as used herein, are intended to refer to a direction toward (proximal) and away from (distal) a user of the device.

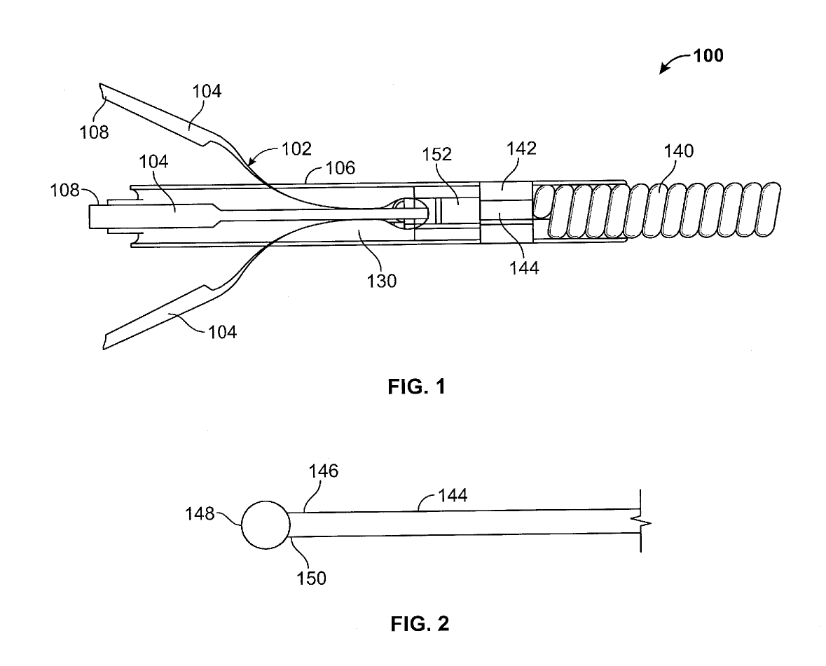

As shown in FIGS. 1-9, a clipping device 100 according to an exemplary embodiment of the invention comprises a clip 102 including a plurality of gripping arms 104 movable within a capsule 106 between an open configuration and a closed configuration. In one exemplary embodiment, as shown in the FIGS. 1-9, the clip 102 includes four arms 104 biased toward an open configuration in which the arms 104 are spread apart from one another so that tissue to be clipped may be received between the arms. The clip 102, however, may include any number of arms 104 which would facilitate omnidirectional closure about target tissue, i.e., three or more arms. For example, the clip 102 may include three, four or six or more arms. Those skilled in the art will also understand that the clip arms 104 may be biased toward the open configuration through properties inherent in some portion of the clip arms themselves or through a separate biasing member. The clipping device 100 is inserted into a body (e.g., through the working channel of a flexible endoscope) with the arms 104 constrained to remain in the closed configuration by the capsule 106. In this closed configuration, distal ends 108 of the arms 104 are brought together minimizing a profile of the device as it is advanced through the body to the target tissue site. Once the clip 102 has been advanced through the body to a desired position adjacent the target tissue, the clip arms 104 are advanced distally out of the capsule 106 so that the arms 104 move to the open configuration. The four arms 104 provide an omnidirectional closure over target tissue eliminating the need to rotate the clip 102 to capture target tissue.

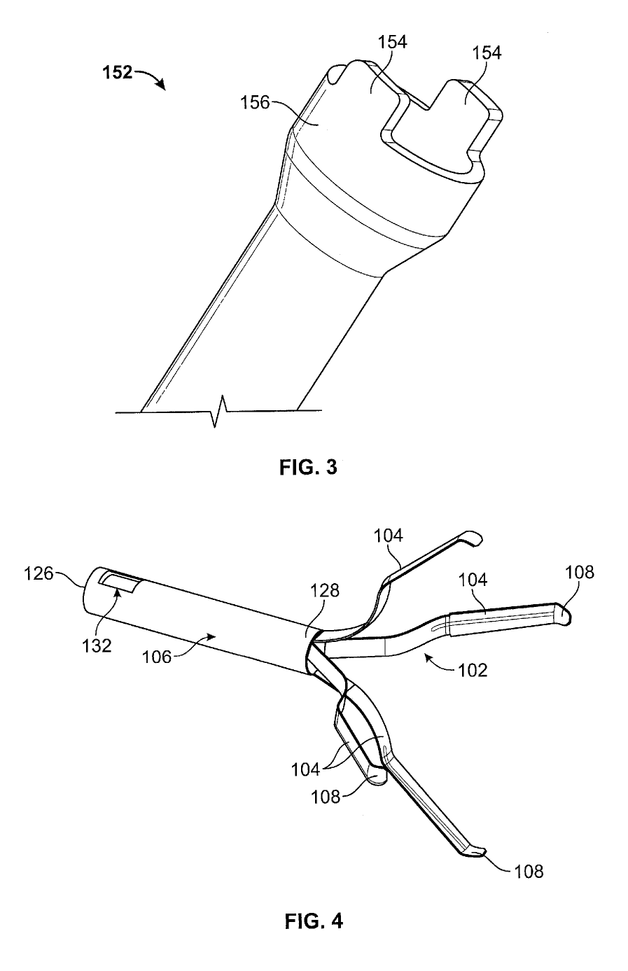

As shown in FIG. 1, the clip 102 and the capsule 106 may be releasably coupled to a proximal portion of the device 100 including a flexible member 140 extending from a proximal end attached to an actuating handle (not shown) to a distal end attached to a bushing 142 which may be releasably coupled to the capsule 106. The flexible member 140 permits insertion of the clipping device 100 through even tortuous paths of the body. The actuating handle at the proximal end may extend out of the body during operation of the device 100. A deployment mechanism of the clipping device 100 may include a control wire 144 and a constraint tube 152. As shown in FIG. 2, the control wire 144 extends through the flexible member 140 from a proximal end attached to the actuating handle to a distal end 146 coupled to a proximal end 110 of the clip 102 to control movement of the clip 102 between the open and closed configurations. The distal end 146 may, for example, include an attachment 148, such as a ball, attached thereto via a joint 150, a portion of the control wire 144 formed to be weaker than the rest of the wire 144 or any other known separable link designed to fail when subject to a predetermine load. Alternatively, the joint 150 may include a linking feature such as, for example, jaws biased toward an open configuration, which is configured to open to release the attachment 148 when the linking feature is received within a relief portion of the capsule 106. The attachment 148 may also be, for example, a T-shaped attachment, a cube, a hook, or any other attachment element which facilitates a coupling with the proximal end 110 of the clip 102. The joint 150 may, for example, be formed as a weld, a thinner portion, a smaller diameter portion, a notched portion, a fatigued portion, a dissimilar material, a hollow portion, or any other suitable connection. When the joint 150 fails, the clip 102 is decoupled from the proximal portion of the device 100 so that the clip 102 may be left in place over the target tissue until the natural healing process has progressed to the point where the tissue is sealed and the clip is sloughed off through natural processes. As shown in FIG. 3. the constraint tube 152 may extend about a portion of the control wire 144 proximal of the attachment 148 and may includes tabs 154 such as a pair of tabs 154 extending from a flared distal end 156 thereof to engage a proximal end of the clip 102, as will be described in further detail below.

The clip 102 shown in FIGS. 4-8 may be integrally formed from a single piece with the arms 104 connected to one another at the proximal end 110 of the clip 102. Alternatively, the arms 104 may be formed separately and joined in any suitable known manner. For example, a proximal end of each of the arms 104 may be connected to a linking member which may be releasably coupled to the control wire 144 via the attachment 148. The linking member grips the attachment 148 of the control wire 144 until the jaws are received within a relief portion of the capsule 106, releasing the attachment 148 therefrom. The arms 104 may be spread equally about a circumference of the clip 102 so that, in the open configuration, the four arms 104 are separated from one another by an angle of about 90° to provide omnidirectional closure. The arms 104 may be sized and configured such that all four arms may be drawn proximally into the capsule 106 in the closed configuration. The arms 104 may be formed of an elastic material such that the arms 104 may revert to a biased open configuration when subsequently moved to the open configuration. Distal ends 108 of the arms 104 may be curved inward and may include teeth 112 to grip target tissue. The teeth 112 according to this embodiment, when in the closed configuration, may form a gap 116 therebetween to minimize trauma to gripped tissue. The gap 116 may be, for example, circular. It will be understood by those of skill in the art, however, that the gap 116 may take any shape so long as sufficient space is left between the teeth to minimize trauma to gripped tissue. Distal ends 108 of the arms 104 may also narrow such that, when the arms 104 are drawn proximally into the capsule 106 and deployed, the clip 102 has a small profile. The clip 102, however, may have any geometry that would provide a small profile upon deployment. The arms 104 may be configured to interlock with one another when compressed within the capsule 106. Alternatively, the arms 104 may be configured to overlap with one another in a predetermined pattern. In one exemplary embodiment, the distal ends 108 of the arms 104 may be staggered in length to allow for better compression of the arms 104 within the capsule 106.

Each of the arms 104 may also include a shoulder 138 positioned along an exterior thereof When the clip 102 is drawn proximally into the capsule 106, the shoulders 138 may contact a distal end 128 of the capsule 106 defining a maximum extent to which the clip 102 may be drawn proximally into the capsule 106. In another embodiment, a shoulder may be positioned along an interior of the capsule 106 rather than on the arms 104 to engage a portion of the arms 104. In yet another embodiment, both the arms 104 and the capsule 106 may include corresponding shoulders which engage one another and define a maximum extent to which the clip may be drawn proximally into the capsule 106. As will be described in more detail below, once the clip 102 has been drawn proximally into the capsule 106 to this maximum extent, further proximally directed actuation of the device, increases tension applied to a control wire 144 until, when a threshold level is exceeded, the clip 102 can be separated from the control wire 144 and permanently affixed to tissue gripped thereby.

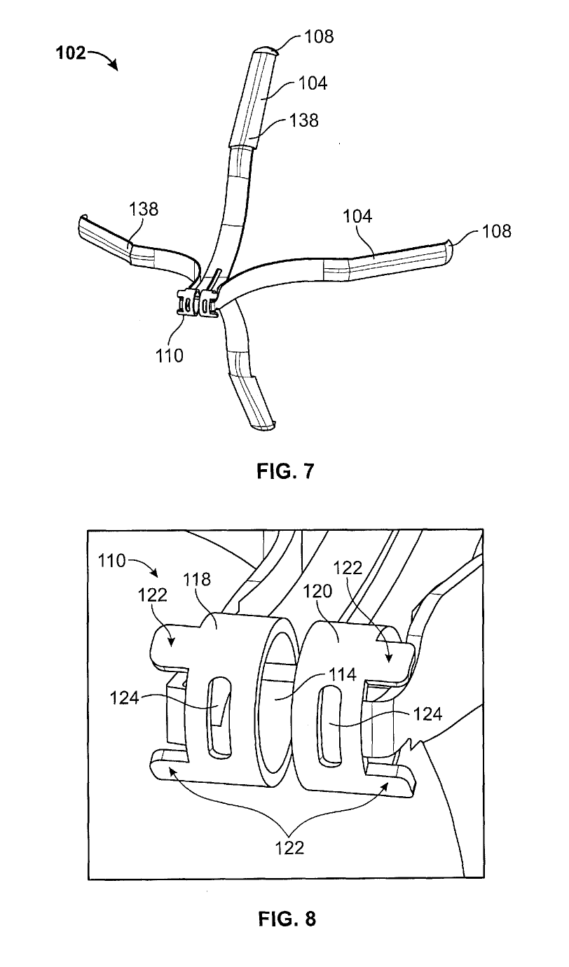

As shown in FIG. 7, the proximal end 110 of the clip 102 may include a space 114 between ends of the arms 104, which is sized and shaped to receive the attachment 148 at the distal end 146 of the control wire 144 which connects the clip 102 to the proximal portion of the device 100. The attachment 148 is capable of being loosely received within the clip to permit the control wire 144 to be rotated independently of the clip 102. Thus, rotation of the control wire 144 does not necessarily translate into a rotation of the clip 102. As discussed above, the attachment 148 may include, for example, a ball, a T-bar, a cube, a hook, etc. The proximal end 110 may further include two laterally separated portions 118, 120, each of which includes a lock tab 122 extending laterally therefrom. In an exemplary embodiment, each of the laterally separated portions 118, 120 may include lock tabs 122 extending therefrom. Each of the laterally separated portions 118, 120 may also include slot 124 engaging a corresponding one of the tabs 154 of the constraint tube 152. The laterally separated portions 118, 120 are biased toward a laterally outward position in which the lock tabs 122 extend away from a centerline of the clip 102. The tabs 154 of the constraint tube 152 may engage the slots 124 to hold the separated portions 118, 120 in a laterally constrained position in which the lock tabs 122 are drawn toward the centerline of the clip 102. In the constrained position, the clip 102 is permitted to move between the open and closed configuration repeatedly, as desired. Once the joint 150 of the control wire 144 fails, however, the tabs 154 can be disengaged from the slots 124 freeing the lock tabs 122 to move laterally outward into engagement with a portion of the capsule 106 to lock the arms 104 in the closed configuration.

The capsule 106 extends from a proximal end 126 to a distal end 128 and may include a lumen 130 extending therethrough. The lumen 130 may be sized and shaped to permit the proximal end 110 of the clip 102 to be slidably movable therewithin and to constrain the arms 104 when the clip 102 is in the closed configuration. The proximal end 126 of the capsule 106 may include windows 132 extending laterally therethrough positioned and shaped to engage the lock tabs 122 when the laterally separated portions 118, 120 are in the laterally outward position, as shown in FIG. 6. Prior to locking and deployment of the clip 102 and capsule 106, however, the windows 132 can engage tabs 136 of the bushing 142 at the distal end of the flexible member 140 to couple the capsule 106 and the clip 102 to the flexible member 140. Thus, the windows 132 can also be sized and shaped to engage corresponding tabs 136 of the bushing.

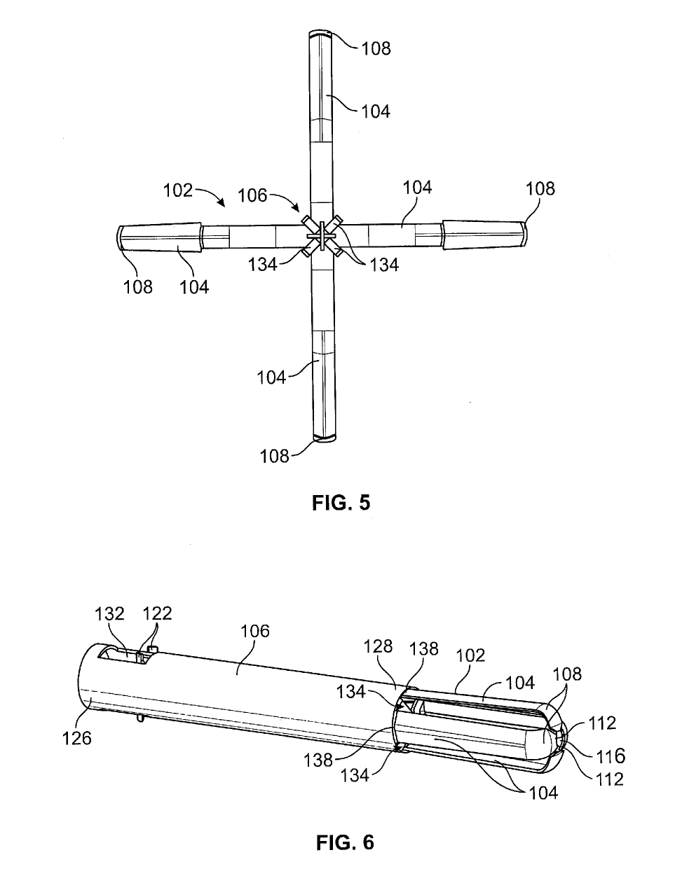

As shown in FIGS. 5 and 6, the distal end 128 of the capsule 106 may include four capsule tabs 134 bent inward at approximately 90° toward a centerline of the capsule 106 to form a substantially X-shape. It will be understood by those of skill in the art, however, that the capsule tabs 134 may be bet inward at any angle toward the centerline of the capsule 106. Each of the capsule tabs 134 may be positioned between an adjacent pair of arms 104 holding the arms 104 in a desired spatial relationship to one another with the arms 104 sliding between the capsule tabs 134. The capsule tabs 134 ensure prevent the clip 102 from rotating relative to the capsule 106 so that the lock tabs 122 remain aligned with the windows 132 during deployment. The capsule tabs 134 can also act as a stop, keeping the proximal end 110 of the clip 102 within the lumen 130 of the capsule 106 at all times. Although this exemplary embodiment specifically describes four capsule tabs 134, it will be understood by those of skill in the art that the capsule 106 may include any number of capsule tabs 134. For example, the number of capsule tabs 134 may correspond to a number of arms 104 of the clip 102 such that each of the arms 104 is positioned between adjacent tabs 134. Alternatively, the capsule 106 may include a number of capsule tabs 134 less than a number of arms 104 so that, although it is not necessary, the clip 102 may be rotated about a longitudinal axis thereof relative to the capsule 106.

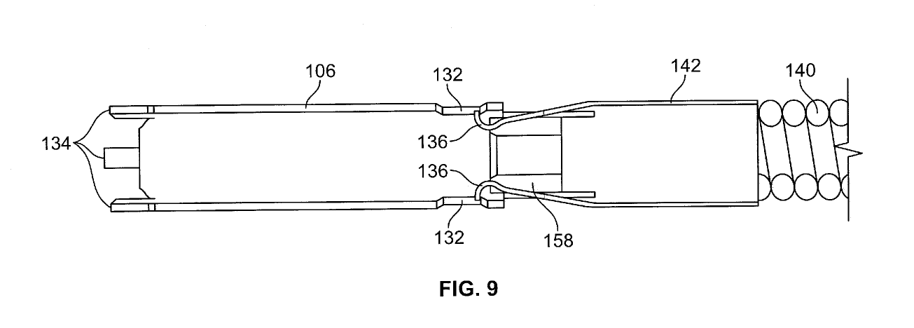

As shown in FIG. 9, the tabs 136 of the bushing 142 are moved radially outward to engage the windows 132 of the capsule 106 when a bushing support 158 is received within a distal end thereof. This locks the bushing 142 to the capsule 106. The tabs 136 are biased toward a centerline of the bushing 142 such that when the bushing support 158 is removed therefrom, the tabs 136 revert to the biased configuration disengaging from the windows 132 and freeing the capsule 106 from the bushing 142 and, consequently, from the rest of the device 100. The bushing support 158 can include an engaging surface which engages radially inner surfaces of the bushing 142 to maintain a position therewithin. For example, the engaging surface may include a groove or pocket in which a radially inward projection of the tabs 136 is received. The bushing support 158 remains within the bushing 142 until the joint 150 of the control wire 144 fails. When the joint 150 fails, the attachment 148 remains within the space 114 of the proximal end 110 while a remaining length of the control wire 142 and the constraint tube 152 connected thereto are drawn proximally until the flared end 156 of the constraint tube 152 contacts a distal end of the bushing support 158, dislodging the bushing support 158 from within the bushing 142. The tabs 136 of the bushing 142 then become disengaged from the windows 132 of the capsule 106, disengaging the capsule 106 from the proximal portion of the device 100.

The clipping device 100 is inserted into the body in the closed configuration until the clip 102 is adjacent the target tissue within the body. The control wire 144 can then be moved distally relative to the capsule 106 to move the clip 102 to the open configuration. Alternatively the capsule 106 may be moved proximally relative to the clip 102 to move the clip 102 to the open configuration. The clip 102 is then positioned so that distal ends 108 of the arms 104 surround a target portion of tissue. As indicated previously, the clip 102 may be moved as often as desired between the open and closed configurations, by moving the control wire 144 distally and proximally relative to the capsule 106, until the target tissue is gripped by the teeth 112 at the distal ends 108, as desired. It will be understood by those of skill in the art that the clip 102 is not required to be rotated to a particular angular orientation relative to the tissue since the plurality of arms 104 provide an omnidirectional closure thereover. The control wire 144 can then be drawn proximally until the shoulders 138 along the arms 104 come into contact with the distal end 128 of the capsule 106 preventing the clip 102 from moving farther proximally into the capsule 106. Once the target tissue has been gripped, as desired, the control wire 144 is drawn farther proximally increasing the tension on the control wire 144 until the joint 150 fails. Alternatively, the joint 150 may fail when a linking member thereof is received within a relief portion of the capsule 106, thereby releasing the attachment 148 of the control wire 144. Failure of the joint 150 results in disengagement of the tabs 154 of the constraint tube 152 from the slots 124 of the clip 102 so that the locking tabs 122 move to the laterally outward position. The flared end 156 moves proximally against the distal end of the bushing support 158 until the bushing support 158 is dislodged from within the bushing 142 so that the tabs 136 thereof are disengaged from the windows 132 of the capsule 106. The locking tabs 122 engage the windows 132 of the capsule 106, locking the clip 102 within the capsule 106. The proximal portion of the device 100 may then be removed from the body, leaving the deployed clip 102 and capsule 106.

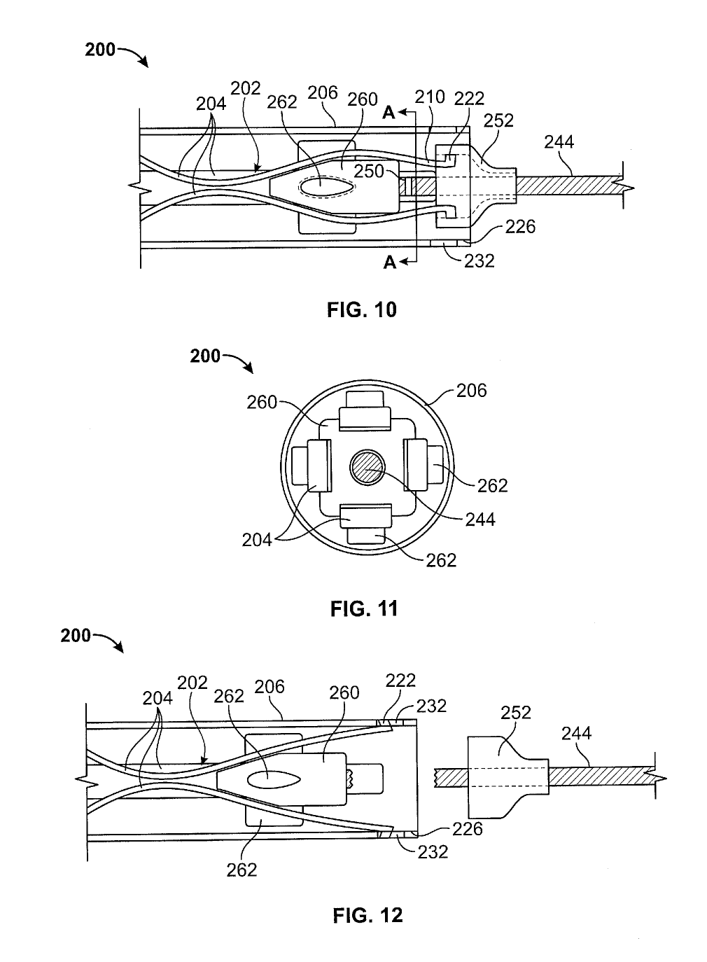

As shown in FIGS. 10-12, a clipping device 200 according to another exemplary embodiment of the present invention is substantially similar to the clipping device 100, comprising a clip 202 including a plurality of arms 204, in this embodiment four arms 204, movable within a capsule 206 between an open configuration, in which the arms 204 are spread apart from one another to receive tissue therebetween, and a closed configuration, in which distal ends 208 are moved toward one another to grip the tissue therebetween. The four arms 204 of the clip 202, however, are not formed as a single piece in which proximal ends 210 are integrally connected to one another, but rather, are formed as four separate clip arms 204, coupled to a core member 260. The core member 260 is coupled to a control wire 244 substantially similar to the control wire 144 permitting the core member and clip 202 to be moved proximally and distally relative to the capsule 206, between the open and the closed configuration. The core member 260 may be connected to the control wire 233 via a joint 250, which may be designed similarly to the joint 150 to fail when subject to a predetermined load. Alternatively, the joint 250 may be designed to fail when jaws thereof are received within a relief portion of the capsule 206. Jaws of the joint 150 may be biased in an open configuration but restricted in a closed configuration to grip an attachment at a distal end of the control wire 144 via an inner surface of the capsule 206 until the control wire 244 is drawn proximally and the joint 150 is received within the relief portion of the capsule 206 to release the attachment. Proximally of the joint 250, along the control wire 244, the device 200 further comprises a constraint member 252 fixedly attached thereto. The constraint member 252 constrains proximal ends 210 of the arms 204 to prevent the proximal ends 210 from engaging the capsule 206 prior to locking and deployment of the clip 202. Similarly to the device 100, the control wire 244 extends through a flexible member to an actuating handle at a proximal end thereof and include a joint designed to fail when subjected to a predetermined load.

The arms 204 of the clip 202 extend from the proximal end 210 to a distal end 208, the proximal end 210 coupled to the core member 260. Proximal portions of the arms 204 may include cut-outs sized and shaped to receive corresponding protrusions 262 of the core member 260 to couple the arms 204 to the core member 260. Proximally of the cut-outs 262, each of the arms 204 may include a locking element 222 such as, for example, a protrusion, tab or hook, extending laterally from an exterior surface thereof to engage a corresponding window 232 extending laterally through a proximal end 226 of the capsule 206, when the clip 202 is deployed. The proximal ends 210 of the arms 204 may be biased radially outward and constrained via the constraint tube 252 such that the locking elements 222 are prevented from engaging the windows 232 until it is desired to lock the clip 202 in the closed configuration and deploy the clip 202.

The device 200 may be used in a manner substantially similar to the device 100. In particular, the device 200 is inserted into the body in the closed configuration until a distal end thereof is adjacent target tissue. The device 200 may then be moved to the open configuration by moving the control wire 244 distally relative to the capsule 206 such that distal ends 208 of the arms 204 extend distally past a distal end of the capsule 206. The clip 202 is then positioned such that distal ends 208 of the open arms 204 surround the target tissue. The control wire 244 is then drawn proximally relative to the capsule 206 to grip the target tissue between the distal ends 208. The clip 202 may be moved between the open and the closed configurations, as desired, until the target tissue has been gripped as desired. Once the target tissue has been gripped as desired, the control wire 244 is drawn farther proximally relative to the capsule 206 until the joint 250 is broken, drawing a remaining portion of the control wire 244 and thereby the constraint member 252 proximally relative to the capsule 206. Severing of the joint 250 releases the proximal ends 210 of the arms 204 such that the locking elements 222 engage the windows 232 and the clip 202 is locked in the closed configuration and deployed.

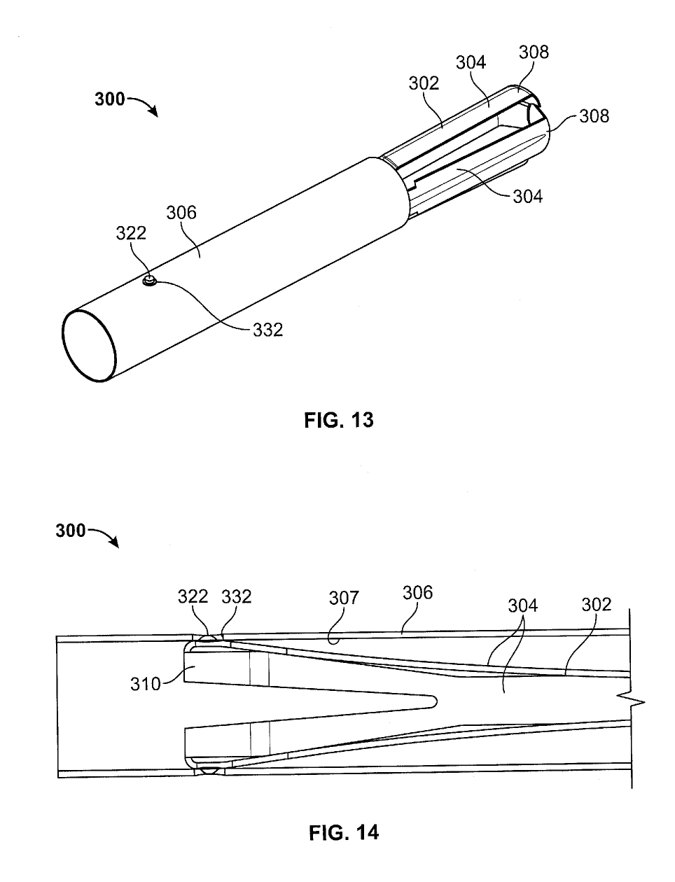

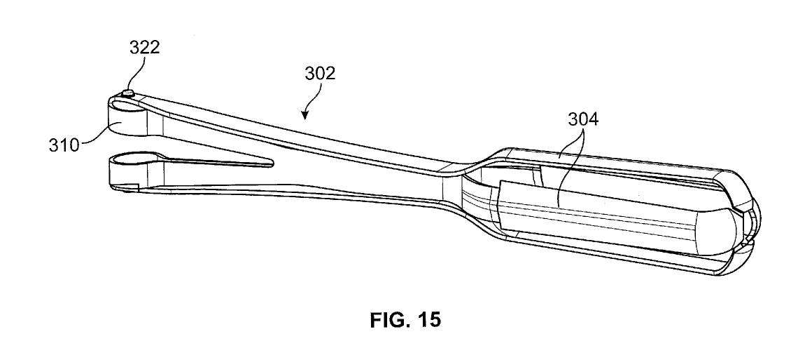

As shown in FIGS. 13-15, a clipping device 300 according to another exemplary embodiment of the present invention may be substantially similar to the clipping devices 100, 200 described above. The clipping device 300, however, comprises a clip 302 including a plurality of clip arms 304, each of the clip arms 304 extending from a proximal end 310 to a distal end 308. The clip arms 304 in this embodiment are connected to one another similarly to the clip 102 of the device 100. Alternatively, the clip arms 304 may be formed as separate elements similarly to the clip 202 of the device 200. The arms 304 are biased radially outward and the proximal end 310 of each of the arms 304 includes a protrusion 322 which, in a locked and deployed configuration, extends laterally from an exterior surface thereof to engage a corresponding cut-out 332 to project laterally through a proximal end of a capsule 306 substantially similar to either of the capsules 106, 206. In an unlocked configuration, the protrusion 322 exerts a radially outward pressure such that the protrusion 322 rides along an interior surface 307 of the capsule as the clip 302 is moved between the open and closed configurations. Once target tissue has been gripped between distal ends 308 of the clip arms 304 as desired, the clip 302 may be drawn proximally relative to the capsule 306 until the protrusions 322 engage the cut-outs 332.

It will be apparent to those skilled in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A tissue clipping device, comprising:
a capsule extending from a proximal end to a distal end and including a lumen extending therethrough;
a clip including at least three arms having proximal and distal ends and being biased in an open configuration and connected to one another at proximal ends thereof, the arms being separated circumferentially about a centerline of the clip, the proximal ends of the clip arms being movable within the lumen of the capsule to move the clip between an open configuration, in which distal ends of the arms are separated from one another to receive tissue therebetween, and a closed configuration in which distal ends of the arms are drawn toward one another to grip the tissue therebetween; and
a tension member releasably coupling the clip to a proximal end of the device which, during use, remains accessible to a user, to control movement of the clip between the open and the closed configurations.

2. The device of claim 1, wherein at least one arm of the clip includes a locking member biased to engage a locking structure of the capsule.

3. The device of claim 2, further comprising a constraint member coupled to the tension member and releasably connected to the clip to maintain the locking member of the arm in a constrained position in which the locking member is prevented from engaging the locking structure of the capsule.

4. The device of claim 3, wherein the tension member is coupled to the clip via a joint designed to release when subject to a predetermined load to separate the clip from the device.

5. The device of claim 4, wherein release of the joint releases the locking member to engage the locking structure of the capsule.

6. The device of claim 1, wherein distal ends of the arms are curved inward toward the centerline of the clip and include teeth to grip tissue.

7. The device of claim 6, wherein, when the clip is in the closed configuration, a gap extends between each of the distal ends.

8. The device of claim 1, wherein the at ns include a shoulder positioned along an exterior thereof such that contact between the shoulder and the distal end of the capsule substantially prevents the clip from being drawn farther proximally into the capsule.

9. The device of claim 1, wherein the distal end of the capsule includes tabs bent inward toward a centerline thereof to hold each of the arms in position.

10. The device of claim 1, wherein the clip is formed as an integral unit.

11. The device of claim 1, wherein proximal ends of the arms are connected to a core member.

12. The device of claim 11, wherein the core member is releasably coupled to the tension member.

13. A method for clipping tissue, comprising the steps of:
inserting a clipping device, in a closed configuration, to a target area within a body, the clipping device including a capsule extending from a proximal end to a distal end and including a lumen extending therethrough and a clip including at least three arms separated from one another circumferentially about a centerline of the clip and connected to one another at proximal ends thereof;
moving the clipping device to an open configuration in which distal ends of the arms are separated from one another to receive target tissue therebetween by moving the proximal end of the clip distally within the lumen via a tension member releasably coupled to the clip, the arms of the clip being biased toward the open configuration such that as the clip extends distally out of the capsule the arms open; and
moving the clip to the closed configuration to grip target tissue between the arms by moving the proximal end of the clip proximally within the lumen via the tension member.

14. The method of claim 13, further comprising deploying the clip by drawing the tensioning member proximally relative to the capsule until a joint of the tension member releases.

15. The method of claim 14, wherein release of the joint frees a constraint member connected to at least one arm of the clip, the constraining member prior to being freed maintaining a locking member of the clip in a constrained position in which the locking member is prevented from engaging a locking structure of the capsule such that, after the constraint member is freed, the locking member engages the locking structure.

16. The method of claim 13, wherein distal ends of the arms are curved inward toward the centerline of the clip and include teeth to grip the target tissue.

17. The method of claim 13, wherein, when the clip is in the closed configuration, the target tissue is received within a gap extending between each of the distal ends.

18. The method of claim 13, wherein the clip is in the closed configuration when a shoulder positioned along an exterior of at least one of the arms contacts the distal end of the capsule.

19. The method of claim 13, wherein tabs at the distal end of the capsule bent inward toward a centerline thereof hold at least one arm in position as the clip is being moved between the open and the closed configurations.

20. The method of claim 13, wherein the clip is formed of an integral unit.

21. The method of claim 13, wherein proximal ends of the arms are connected to a core member, which is releasably coupled to the tension member.

22. A tissue clipping device, comprising:
a capsule extending from a proximal end to a distal end and including a lumen extending therethrough, a clip including at least three arms biased toward an open configuration and connected to one another at proximal ends thereof, the arms being equidistantly separated from one another circumferentially about a centerline of the clip, the proximal ends being slidable within the lumen of the capsule to move the clip between the open configuration, in which distal ends of the arms are separated from one another to receive tissue therebetween, and a closed configuration in which distal ends of the arms are moved toward one another to grip tissue therebetween; and
a tension member releasably coupling the clip to a proximal end of the device which, during use, remains accessible to a user, to control movement of the clip between the open and the closed configurations.
